(19) United States
(12) Patent Application Publication
(10) Pub. No.: US 2023/0363927 A1
(43) Pub. Date: Nov. 16, 2023

(54) PREPACKAGED SACROILIAC JOINT IMPLANT WITH PREPACKED BONE GROWTH FACTOR

(71) Applicant: PTL Opco, LLC, Tampa, FL (US)

(72) Inventors: Sean LaNeve, Tampa, FL (US); Dwayne Polzer, Lutz, FL (US); Daniel Newman, Edina, MN (US)

(73) Assignee: PTL Opco, LLC, Tampa, FL (US)

(21) Appl. No.: 18/307,730

(22) Filed: Apr. 26, 2023

Related U.S. Application Data
(60) Provisional application No. 63/335,115, filed on Apr. 26, 2022.

(51) Int. Cl. A61F 2/44; A61F 2/28; A61L 27/54; A61F 2/00; A61L 27/36; A61L 27/26; A61L 27/58

(52) U.S. Cl. CPC A61F 2/447; A61F 2/0095; A61F 2/2846; A61L 27/26; A61L 27/3608; A61L 27/54; A61L 27/58; A61F 2002/3068

(57) ABSTRACT

An allograft implant for fusing a sacroiliac joint. The allograft implant has a body with a graft window disposed therein. A bone growth factor is prepacked into the graft window at the manufacturing stage, prior to packaging and shipping. The implant with the prepacked bone growth factor is hermetically sealed in a package. The allograft implant may be classified as “human cells, tissues, and cellular and tissue-based product (HCT/P),” while the bone growth factor may be classified as a non-HCT/P. The implant may comprise a dissolvable or bioabsorbable membrane for retaining the growth factor inside the graft window. The graft window may comprise a scaffold for retaining a putty-like or a sponge-like bone growth factor.

### PRIORITY CLAIM

This non-provisional patent application claims priority to U.S. Provisional Application No. 63,335,115 filed on Apr. 26, 2022.

### BACKGROUND

### (1) Field of Endeavor

The present invention relates generally to the field of sacroiliac joint fusion procedures, and more particularly, to a prepackaged allograft implant having bone fusion material incorporated therein and packaged as a single unit.

### (2) Description of Related Art

The sacroiliac joint (“SI joint”) is located at the interface between the sacrum and ilium bones in a human’s pelvic area. The SI joint includes strong ligaments that permit only slight movement between the sacrum and the ilium. The sacrum is connected to the base of the spine, and each ilium is connected to the top of the leg and hip area. Thus, the SI joint is the interface between a human’s upper body and lower body.

Dysfunction in the SI joint is a common problem of back pain. In fact, over 25% of back pain is caused by SI joint dysfunction. Even a properly functioning SI joint can become painful after certain types of spinal procedures. For example, over 75% of lumbar fusion surgeries lead to SI joint pain. Often, SI joint pain or dysfunction is addressed by fusing the SI joint, and many past procedures exist for doing so. Past SI joint fusion procedures involve installation of complex implant devices, such as bone anchors, fusion devices, and multi-component implants. These procedures involve complex devices, such as drills and drill bits, and multi-component dilators, braces, and anchor installation devices. As a result, fusion procedures using these devices are complex and time consuming, often leading to suboptimal results.

Another complication of SI joint fusion is that promoting fusion of the sacrum and ilium can be difficult to achieve without the use of a bone fusion agent or material to promote osteogenesis. Such materials include the well-known autograft and allograft materials, as well as, demineralized bone matrix, xenograft, and a variety of natural bone graft substitutes. Synthetic materials, such as bioceramics, polymers, and osteoinductive factors can also be used as the bone fusion material. Common synthetic bone graph substitutes include calcium sulfate, calcium phosphate (CaP), ceramics (including hydroxyapatites (Hap), tricalcium phosphate (TCP), biphasic calcium phosphate (BCP)), CaP cements, bioactive glass, or combinations thereof. Bone growth factors may include bone morphogenetic proteins (BMPs), fibroblast growth factors (FGFs), vascular endothelial growth factor (VEGF), parathyroid hormone (PTH), and platelet-rich plasma (PRP). Graft substitutes of any variety may also contain bioinorganic ions, including magnesium (Mg), strontium (Sr), silicon (Si), zinc (Zn), copper (Cu), lithium (Li), and cobalt (Co).

In prior applications, bone fusion materials were combined with the implant during surgical procedures. Before, during, or after placement of the implant in the SI joint, the bone fusion materials are packed in and around the implant. This process requires the surgeon to place, pressure, prod, and manipulate the bone fusion material in an attempt to properly locate the fusion material inside the SIjoint and around the implant. Even then, proper placement of the fusion material can be elusive, thus rendering the fusion material ineffective to promote osteogenesis.

The present device seeks to overcome these problems by providing an allograft implant having bone fusion material that is incorporated into the implant during the manufacturing or sterilization process rather than during the surgical procedure process.

### SUMMARY OF THE INVENTION

In an embodiment, the invention pertains to a prepackaged implant for fusing a sacroiliac joint (SI joint). The prepackaged implant has a body having a first face and a second face disposed in an opposing relationship to one another. When the body of the implant is positioned with the SI joint, the first face is configured to make abutting contact with a sacrum and the second face configured to make abutting contact with an ilium. In an embodiment, the body of the implant is composed of human cells, tissues, or cellular or tissue-based products (HCT/Ps), as defined by United States Food and Drug Administration (FDA).

The body of the implant includes a graft window, which opens to the first and the second faces. Accordingly, the graft window provides a passage between the first and the second faces through the body of the implant.

In an embodiment, a non-HCT/P bone growth factor is prepacked within the graft window. The prepackaged implant further includes a package hermetically sealing the body of the implant and the non-HCT/P bone growth factor prepacked into the graft window. The non-HCT/P bone growth factor is prepacked into the graft window prior to sealing the implant within the package.

In an embodiment, the non-HCT/P bone growth factor comprises demineralized bone matrix (DBM) fibers and a carrier, which may be collagen. At least some of the DBM fibers may be positioned with the graft window such that they traverse the graft window. In this manner, when the implant is placed into the SI joint, the DBM fibers traversing the graft window are configured to connect the sacrum and the ilium, thereby facilitating ontogenesis. The non-HCT/P bone growth factor may be freeze-dried, and the DBM fibers may be configured expand when the implant is placed into the SI joint.

In an embodiment, the prepackaged implant constitutes a medical device under the FDA classification and is compliant with the FDA regulations governing HCT/Ps.

The prepackaged implant may include a membrane affixed to the first face and/or the second face of the body of the implant and configured to retain the non-HCT/P bone growth factor within the graft window. The membrane may be composed of a material that is configured to be dissolved or bioabsorbed when the implant is placed into the SI joint. The body of the implant may include a retaining structure disposed within the graft window to retain the non-HCT/P bone growth factor within the graft window.

In an embodiment, the invention pertains to a method of performing a medical procedure for a SI joint fusion. According to this method, a surgeon receives a surgical instrument kit and a package containing a prepackaged implant. The implant has a body composed of human cells, tissues, or cellular or tissue-based products (HCT/Ps). The body has a graft window that is prepacked with a non-HCT/P bone growth factor. A surgeon opens the package and removing therefrom, as a single unit, the body of the implant and the non-HCT/P bone growth factor prepacked into the graft window. Then, the surgeon inserting, as the single unit, the body of the implant with the non-HCT/P bone growth factor prepacked into the graft window into the SI joint. The body of the implant and the non-HCT/P bone growth factor remain with the SI joint, thereby causing the SI joint fusion.

### BRIEF DESCRIPTION OF THE DRAWINGS

FIG. 1 shows the posterior view of a typical human pelvis.

FIG. 2 is a perspective view of an embodiment of an allograft implant for insertion into the SI joint.

FIG. 3 shows an alternate view of one embodiment of an allograft implant.

FIG. 4 shows an insertion end of one embodiment of an implant inserter.

FIG. 5 shows the implant inserter of FIG. 4 with the allograft implant of FIGS. 1-3 seated in the tines.

FIG. 6 is a side view of one embodiment of an allograft implant.

FIG. 7 is a cross-sectional view B-B of the allograft implant of FIG. 6.

FIG. 8 is a top view of one embodiment of an allograft implant.

FIG. 9 is a rear view of one embodiment of an allograft implant.

FIG. 10 is a front view of one embodiment of an allograft implant.

FIG. 11 is a perspective view of an embodiment of an allograft implant having an intermediate support.

FIG. 12 is perspective view of one embodiment of the allograft implant having an intermediate support located in the graft window.

FIG. 13 shows the allograft implant of FIGS. 2 and 3 with exemplary bone growth factor installed in the graft window.

FIG. 14 shows the allograft implant of FIG. 12 with exemplary bone growth factor installed in the graft window.

FIG. 15 shows the allograft implant of FIG. 11 with exemplary bone growth factor installed in the graft window.

FIG. 16 shows the allograft implant of FIG. 13 with one embodiment of a bioabsorbable membrane installed for retention of the bone growth factor in the graft window.

FIG. 17 shows the allograft implant of FIG. 16 with the bioabsorbable membrane partially pulled back.

FIG. 18 shows the allograft implant of FIGS. 2 and 3 with scaffolding installed inside the graft window.

### DETAILED DESCRIPTION OF THE PREFERRED EMBODIMENTS

With reference to the drawings, the improved allograft implant for fusing a sacroiliac joint (“SI joint”) now will be described with regard for the best mode and the preferred embodiment. The embodiments disclosed herein are meant for illustration and not limitation of the invention. An ordinary practitioner will appreciate that it is possible to create many variations of the following embodiments without undue experimentation.

The allograft implant 5 and bone fusion material 25 described herein are used primarily for fusing a sacroiliac (SI) joint in the pelvis of a human. The implant body is made of allograft and therefore is classified by the United States Food and Drug Administration (FDA) as “human cells, tissues, and cellular and tissue-based products (HCT/Ps),” which are defined as human cells or tissues intended for implantation, transplantation, infusion or transfer into a human recipient. Currently, implants made of pure HCTP/Ps are subject only to regulation under section 361 of the Public Health Service Act (PHS Act).

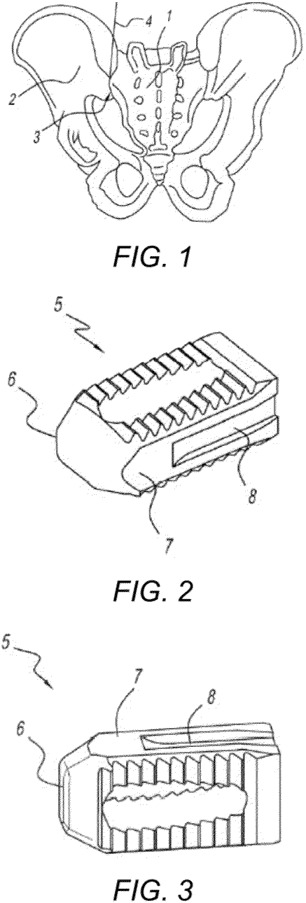

Referring to FIGS. 1-3, an allograft implant 5 is placed in the soft tissue of the SI joint 3 between the sacrum 1 and the ilium 2 of the pelvis. For the purpose of reference, the SI joint plane as used herein means the general plane of the SI joint 3 as defined by the abutting, articular surfaces of the sacrum 1 and ilium 2. The implant 5 provides a matrix for bone healing across the SI joint, thereby fusing the sacrum 1 and ilium 2 together. Referring to FIGS. 2 and 3, one embodiment of the implant 5 generally comprises a nose 6 and at least one pair of opposing lateral sides 7, each comprising a groove 8 that is disposed at least partially along the length of each such lateral side 7.

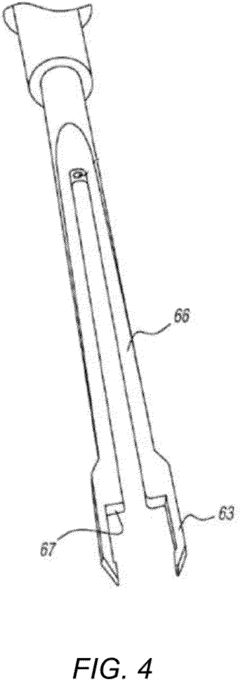

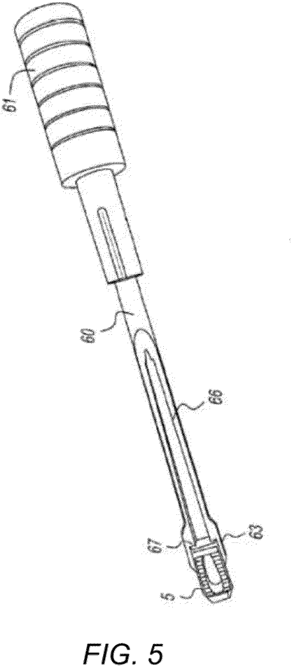

The grooves 8 are disposed for receiving tines 63 on an implant inserter 60. Referring to FIGS. 4-5, one embodiment of an implant inserter 60 comprises a handle 61 and an implant insertion end. The implant insertion end has a pair of tines 63 for holding the implant 5 during the process of inserting the implant 5 into the SI joint 3. Each tine 63 is supported by a tine shaft 66, which terminates at a shoulder 67. The tine shafts 66 provide flexibility such that the implant 5 is removably retained between the tines 63 with the shoulder 67 abutting against the implant 5 (see FIG. 5). For example, in one embodiment the width of the implant 5 between the opposing grooves 8 is slightly larger than the space between the respective tines 63 such that when the implant 5 is seated in the implant insertion end, the respective tines 63 are pushed slightly apart by the grooves 8. This causes a slight amount of friction between the tines 63 and the grooves 8, thereby releasably retaining the implant 5 in the implant insertion end.

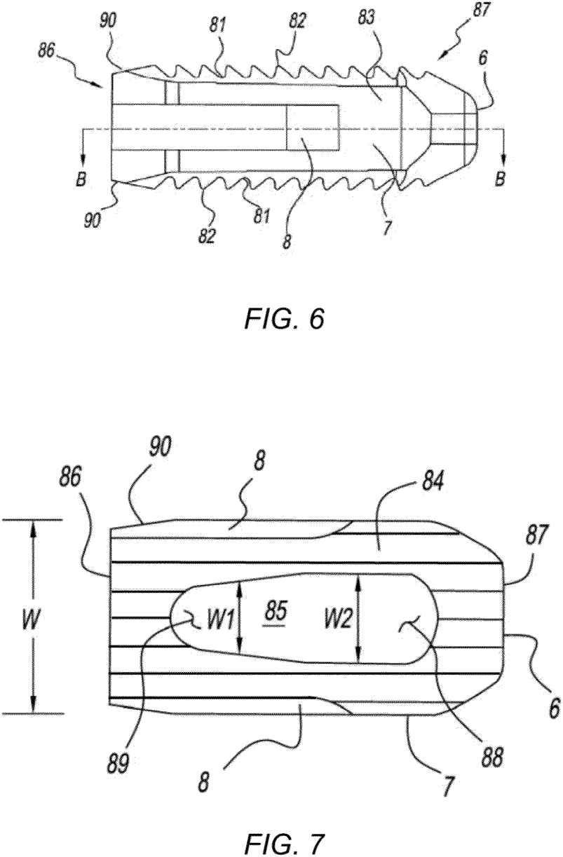

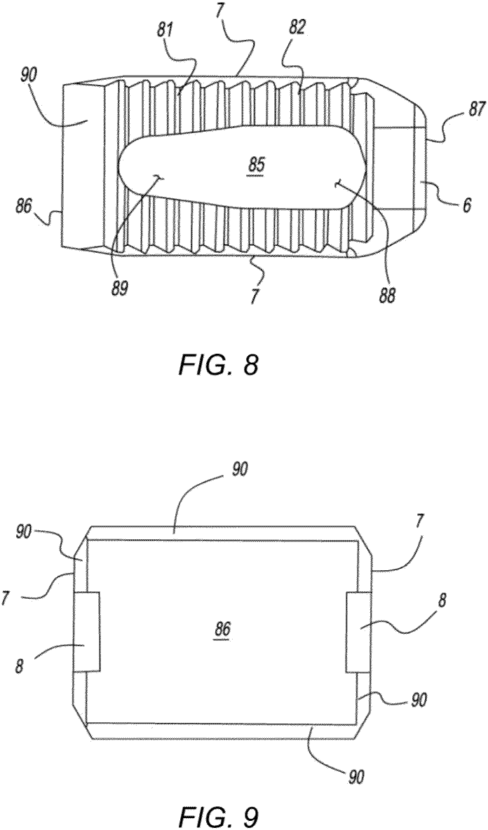

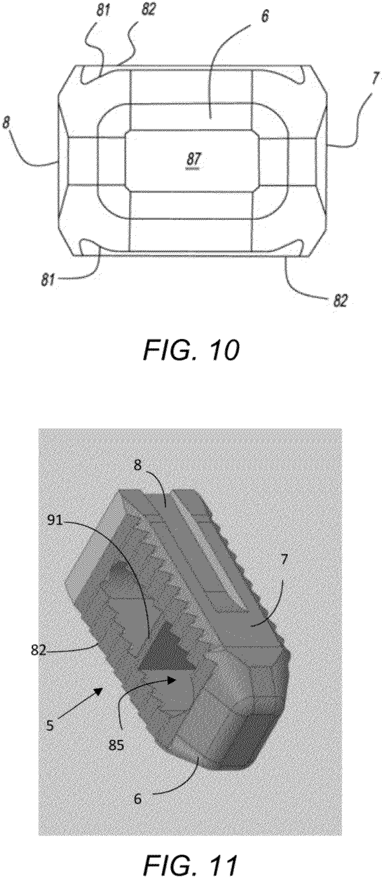

Referring to FIGS. 6-10, the implant 5 generally has two opposing faces 81. When the implant 5 is inserted into the SI joint 3, one opposing face 81 is disposed against the sacrum 2, and the other opposing face 81 is disposed against the ilium 3. The opposing faces 81 may comprise one or more anti-migration features 82, such as teeth, ridges, nubs, barbs, or other surface textures which resist movement of the implant inside the SI joint 3 by increasing surface friction between the surface of the implant 5 and the abutting articular surface of the sacrum 1 or ilium 2.

In one embodiment, the implant 5 comprises a body 83 having a proximal end 86, a distal end 87, and a length disposed therebetween, the distal end having a rounded nose 6. The body 83 further comprises two sides 7, each side 7 comprising a groove 8 beginning at the proximal end 86 of the body 83 and continuing along each of the two sides 7 for at least part of the length, the distance between the two sides 7 defining a width W of the body (see FIG. 7).

The implant 5 further comprises a central graft window 85 that enables fusion of the SI joint 3 to occur through the implant 5. The graft window 85 is disposed between each of the opposing faces 81, the graft window 85 providing passage through the body 83 between the two opposing faces 81. The portion of the body 83 located between the graft window 85 and each of the sides 7 defines a wall 84.

When viewed in cross section (see FIG. 7), the area of the graft window 85 is about 35% to about 40% of a first cross-sectional area of the body, which is the area of the total implant 5 as shown by the hatched area in FIG. 7. The area of the window 85 is about 60% of the total area of each opposing face 81 that makes contact with the bone, whether the sacrum 1 or the ilium 2. These ratios provide a strength-to-contact area relationship that produces an unexpected enhancement to the performance of the implant 5 as a promoter of SI joint 3 fusion. In these ratios, the implant 5 provides enough strength so that it is not crushed inside the SI joint 3, and it provides a large enough graft window 85 to expedite successful fusion of the SI joint 3 across and through the implant 5.

In one embodiment, the graft window 85 has a proximal portion 89 located in proximity to the grooves 8, and a distal portion 88, the proximal portion 89 having a width W1 that is less than a width W2 of the distal portion 88 such that the wall 84 maintains a minimum thickness in a range of about 17% to about 20% of the width W of the body 83.

In the foregoing embodiments, it is preferable, but not required, that the graft window 85 is unobstructed by internal or intermediate supports. Such internal or intermediate supports are sometimes used in allograft implants to provide structural support to the implant. However, these internal or intermediate supports obstruct bone fusion from occurring through the window. As such, the graft window 85 may provide an open passage through the body 83 between the opposing faces 81. The open passage may be rectilinear or curvilinear. The proximal end 86 of the body 83 may further comprise a taper 90 that reduces a second cross-sectional area of the body 83, where the second cross-sectional area is in a plane substantially perpendicular to the length of the body 83.

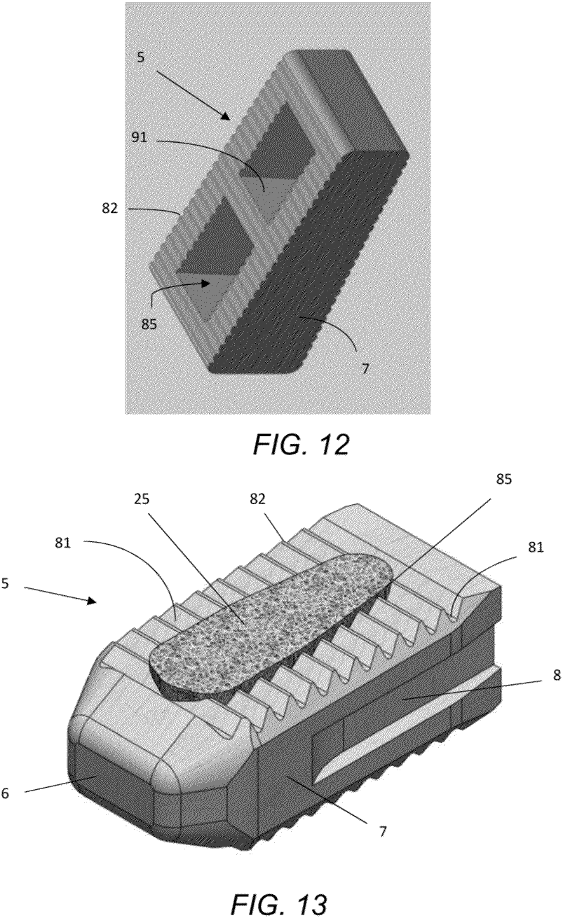

In another embodiment, shown in FIG. 11, the implant 5 has an intermediate support 91 such as a divider, wall, strut, or other similar member. The intermediate support 91 provides further structural support to the allograft implant 5 to resist the crushing force exerted on the implant 5 in the SI joint 3. FIG. 12 shows another embodiment of an implant 5 with an intermediate support 91, without the grooves 8 in the lateral sides 7.

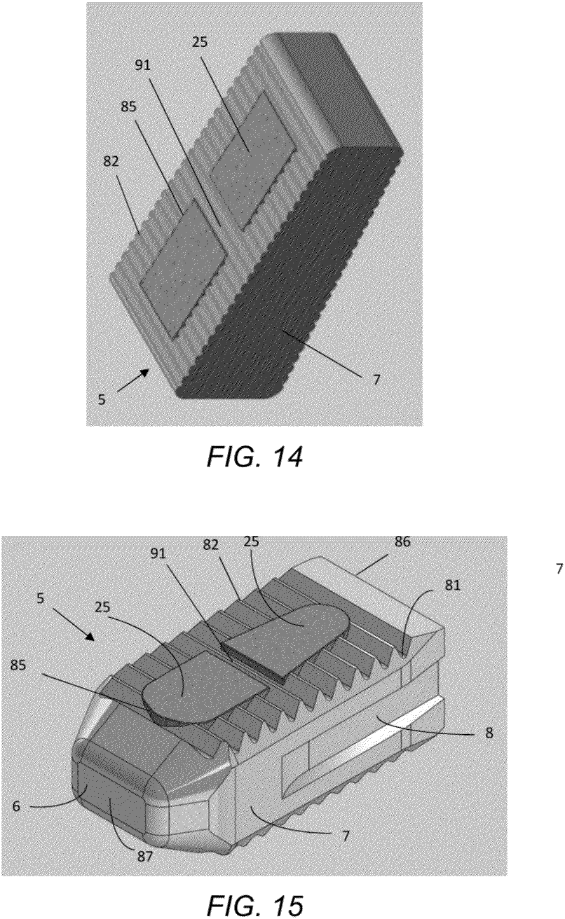

Referring to FIGS. 13 and 14, the bone growth factor 25 is installed in the graft window 85. The bone growth factor 25 is installed in the allograft implant 5 at the manufacturing stage, prior to packaging or delivery for the surgical procedure. In an embodiment, the bone growth factor 25 is a non-HCT/P material and is classified by the FDA as a “medical device.” The bone growth factor 25 may comprise synthetic substances or biological products that are not solely HCT/P (sometimes referred to as “1271 Regulated Tissues,” which are regulated under 21 CFR part 1271). When the non-HCT/P bone growth factor 25 is prepacked into the HCT/P allograft implant 5 at the manufacturing stage, rather than in a surgical room, the resultant combined product is classified by the FDA as a medical device, which is regulated under section 351 of the PHS Act.

Because the prepackaged implant comprises the HCT/P allograft implant body 5 and the non-HCT/P bone growth factor 25, the prepackaged implant must be compliant with the section 361 of the PHS Act (which regulates HTC/Ps) and must also be compliant with the section 351 of the PHS Act (which regulates medical devices). Because of these compounded regulatory burdens, the prepackaged implant 5 constitutes a sharp departure from the current state of the art, according to which allograft implants 5 and bone growth factor 25 are produced, packaged, and delivered to a surgical room separately, such that the HCT/P implant body must only comply with the section 361, while the bone growth factor must only comply with the section 351.

In the current state of the art, because the allograft implant and the gone growth factor are provided separately, the surgeon performing the medical procedure must pack the bone growth factor 25 into the implant body 5 during the surgery. Such manual packing process is prone to human error, which can lead to insufficiently and/or inconsistently packed implants, mishandling or dropping of the implant, breach of sterility, etc.

The current invention eliminates these concerns because the bone growth factor 25 is prepacked into the graft window 85 of the implant 5 at the manufacturing stage using an automated and precise packing process. The prepackaged allograft implant 5 is provided to a surgeon as a single prepackaged unit comprising the implant body 5 and the bone growth factor 25 prepacked into the graft window 85. Thus, when performing a surgical procedure, a surgeon simply opens the package and removes the implant 5 with the prepacked bone growth factor 25 form the package as a single unit. The implant 5 with the bone growth factor 25 prepacked therein is then coupled to the implant inserter 60 and is inserted as a single unit into the SI joint 3, without requiring any additional handling.

As described above, in an embodiment, the bone growth factor 25 is a non-HCT/P bone growth factor, and therefore is classified by the FDA as a medical device. The bone growth factor 25 may include demineralized bone matrix (DBM) fibers and a carrier, which may comprise collagen. The combination of the DBM fibers and collagen results in a putty-like formable substance. The carrier may be synthetic, biologic, or a combination thereof. In either embodiment, because bone growth factor 25 is classified as non-HCT/P, the prepackaged allograft implant 5 with the non-HCT/P bone growth factor 25 is classified by the FDA as a medical device and must be compliant with both sections 361 and 351 of the PHS Act. In some embodiments, the implant body 5 may be composed of a non-HCT/P material, for example, titanium, PEEK, PEKK, etc.

The growth factor 25 is incorporated into the implant in one of three embodiments, as follows:

1. The bone growth factor 25 installed in a central window 85 of the implant 5 (see FIG. 13).

2. The bone growth factor 25 installed in a recess, cavity, indent, bore hole, or other protrusion into the allograft implant 5.

3. The bone growth factor 25 infused or impregnated into the porous voids of the allograft bone material.

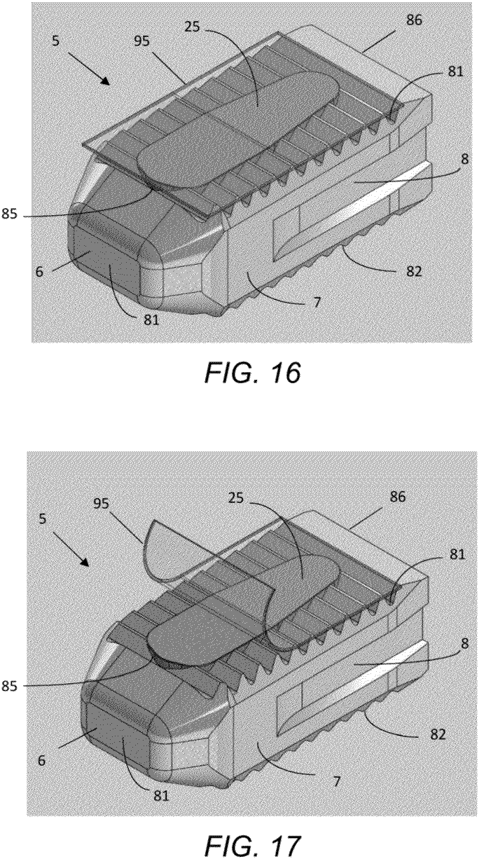

The implant 5 has a bone growth factor 25 installed or incorporated at the time of manufacturer in a pre-packaged manner so that a surgeon does not have to separately combine the growth factor 25 with the implant 5 during the surgical procedure. FIGS. 13-15 show embodiments of how the bone growth factor 25 is pre-packed or pre-loaded into embodiments of the graft window 85. In one embodiment, shown in FIGS. 16 and 17, the synthetic growth factor 25 can be retained in place by a dissolvable or bioabsorbable membrane 95 or by a sponge-like material. The membrane 95 retains the growth factor 25 inside the graft window 85 during the packaging, shipping, and surgical processes. After the implant 5 is installed in the SI joint 3, this membrane dissolves or is absorbed into the patient’s body, thereby releasing the bone growth factor 25 for availability in the fusion process.

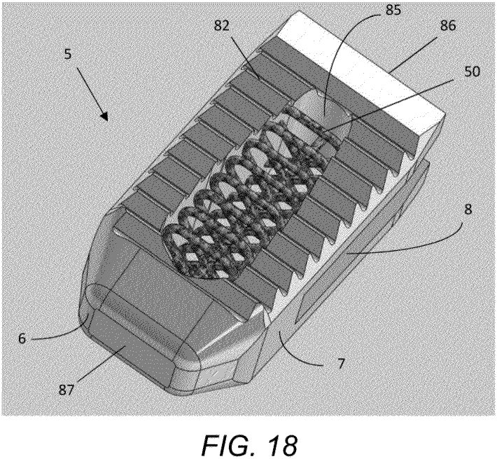

In another embodiment, shown in FIG. 18, the growth factor 25 takes the form of a putty or sponge, and it is held inside the graft window 84 by a scaffold 50. In this embodiment, the growth factor 25 is packed inside the graft window 85 and around the scaffold 50, which provides a retention structure for framework for retaining the sponge-like or putty-like growth factor 25 inside the graft window 85. The growth factor 25 is installed or packed at the manufacturing stage, prior to packaging and shipping.

In another embodiment, bore holes are installed through the implant 5 between the opposing faces 81. The bore holes are then pre-loaded with the bone growth factor 25 prior to packaging and delivery of the implant 5 for the surgical procedure.

Finally, in one embodiment, the bone growth factor 25 is infused into the porous voids of the allograft material of the implant 5, again at the manufacturing level prior to delivery for the surgical procedure.

The foregoing embodiments are merely representative of the SI joint fusion instruments and implant, and not meant for limitation of the invention. For example, persons skilled in the art would readily appreciate that there are several embodiments and configurations of anti-migration elements, grooves, and other features that will not substantially alter the nature of the allograft implant. Consequently, it is understood that equivalents and substitutions for certain elements and components set forth above are part of the invention described herein.

What is claimed is:

1. A prepackaged implant for fusing a sacroiliac joint, comprising:
a body having a first face and a second face disposed in an opposing relationship to one another, the first face configured for abutting contact with a sacrum and the second face configured for abutting contact with an ilium when the body of the implant is inserted into the sacroiliac joint, wherein the body of the implant is composed of human cells, tissues, or cellular or tissue-based products (HCT/Ps);
a graft window disposed within the body of the implant and opening to the first and the second faces, whereby the graft window provides a passage between the first and the second faces through the body of the implant;
a non-HCT/P bone growth factor prepacked within the graft window; and
a package hermetically sealing the body of the implant and the non-HCT/P bone growth factor prepacked into the graft window thereof, wherein the non-HCT/P bone growth factor is prepacked into the graft window prior to sealing the implant within the package.

2. The prepackaged implant of claim 1, wherein the non-HCT/P bone growth factor comprises demineralized bone matrix (DBM) fibers and a carrier.

3. The prepackaged implant of claim 2, wherein the carrier is collagen.

4. The prepackaged implant of claim 2, wherein at least some of the DBM fibers traverse the graft window and are configured to connect the sacrum and the ilium when the implant is positioned within the sacroiliac joint.

5. The prepackaged implant of claim 1, wherein the non-HCT/P bone growth factor is freeze-dried.

6. The prepackaged implant of claim 5, wherein the DBM fibers are configured to expand when the body of the implant is placed into the sacroiliac joint.

7. The prepackaged implant of claim 1, wherein the body of the implant and the prepacked non-HCT/P bone growth factor collectively constitute a medical device under United States Food and Drug Administration (FDA) classification.

8. The prepackaged implant of claim 7, wherein the body of the implant is compliant with the FDA regulations governing the HCT/Ps.

9. The prepackaged implant of claim 1, further comprising a membrane affixed to the first or the second face of the body of the implant and configured to retain the non-HCT/P bone growth factor within the graft window.

10. The prepackaged implant of claim 9, wherein the membrane is configured to be dissolved or bioabsorbed when the implant is placed into the sacroiliac joint.

11. The prepackaged implant of claim 1, further comprising a retaining structure disposed within the graft window, wherein the retaining structure is configured to retain the non-HCT/P bone growth factor within the graft window.

12. A method of performing a medical procedure for a sacroiliac joint fusion, comprising:
receiving a surgical instrument kit and a package containing an implant, wherein the implant has a body with a graft window disposed therein, wherein the body is composed of human cells, tissues, or cellular or tissue-based products (HCT/Ps), and wherein the graft window is prepacked with a non-HCT/P bone growth factor;
opening the package and removing therefrom, as a single unit, the body of the implant and the non-HCT/P bone growth factor prepacked into the graft window thereof; and
inserting, as the single unit, the body of the implant with the non-HCT/P bone growth factor prepacked into the graft window thereof into the sacroiliac joint, whereby the body of the implant and the non-HCT/P bone growth factor remain with the sacroiliac joint, thereby causing the sacroiliac joint fusion.

13. The method of claim 12, wherein the non-HCT/P bone growth factor comprises demineralized bone matrix (DBM) fibers and a carrier.

14. The method of claim 13, wherein the carrier is collagen.

15. The method of claim 13, wherein at least some of the DBM fibers traverse the graft window and are configured to connect the sacrum and the ilium when the body of the implant is positioned within the sacroiliac joint.

16. The method of claim 12, wherein the non-HCT/P bone growth factor comprises a synthetic substance.

17. The method of claim 12, further comprising a membrane affixed to the first or the second face of the body of the implant and configured to retain the non-HCT/P bone growth factor within the graft window.

18. The method of claim 12, further comprising a retaining structure disposed within the graft window, wherein the retaining structure is configured to retain the non-HCT/P bone growth factor within the graft window.

19. The method of claim 12, wherein the body of the implant and the prepacked non-HCT/P bone growth factor collectively constitute a medical device under United States Food and Drug Administration (FDA) classification.

20. The method of claim 19, wherein the body of the implant is compliant with the FDA regulations governing HCT/Ps.
